(19) United States
(12) Patent Application Publication
(10) Pub. No.: US 2008/0058859 A1
(43) Pub. Date: Mar. 6, 2008

(54) Medical Devices Utilizing Modified Shape Memory Alloy

(76) Inventor: Andrzej J. CHANDUSZKO, Chandler, AZ (US)

Correspondence Address: WILMERHALE/BOSTON, 60 STATE STREET, BOSTON, MA, 02109, US

(21) Appl. No.: 11/849,019

(22) Filed: Aug. 31, 2007

Related U.S. Application Data
(62) Division of application No. 10/702,717, filed on Nov. 6, 2003.
(60) Provisional application No. 60/424,086, filed on Nov. 6, 2002.

(51) Int. Cl. A61M 29/00

(52) U.S. Cl. 606/200

(57) ABSTRACT

A medical device made from a shape memory alloy has portions with a first recovery force, and other portions with a second recovery force in desired locations, such as ends that contact portions of the body, such that the second recovery force is less than the first recovery force.

### CROSS-REFERENCE TO RELATED APPLICATION

This application is a Divisional of U.S. patent application Ser. No. 10/702,717, entitled Medical Devices Utilizing Modified Shape Memory Alloy, filed Nov. 6, 2003, which claims priority from provisional application No. 60/424,086, filed Nov. 6, 2002, both of which are expressly incorporated by reference.

### BACKGROUND OF THE INVENTION

Shape memory alloys, such as nitinol (a nickel-titanium alloy that may be doped with other additives such as chromium), are used in a number of medical devices, such as stents, guidewires, blood-clot filters, catheters, and septal occluders. As is known, nitinol can be used in its austenitic phase to form a device. The device is loaded into a catheter in a compressed form, and regains its shape with good stiffness and recovery force at body temperature.

### SUMMARY OF THE INVENTION

A medical device made from a shape memory alloy has portions with a first recovery force, and other portions with a second recovery force in desired locations, such as ends that contact portions of the body, such that the second recovery force is less than the first recovery force. For example, in a device that has nitinol wire loops that come into contact with an artery, heart wall, or other part of the body, the loops can have different recovery force from adjacent portions to reduce any trauma. In some devices, it may be desirable to soften the ends of the device at the device/tissue interface to minimize edge effects (such as in a stent), or in a septal occluder where the edges of the device directly contact a portion of the body. In still other devices, it may be desirable to have a middle portion with less recovery force.

The difference in recovery force can arise from treating certain sections by starting with a relatively low recovery force (softer) wire and stiffening selected sections to produce a stiffer wire, or starting with a stiffer wire and softening certain sections. A softening (or stiffening) process can be performed through some processing of the device, a portion of the device, or a starting material used to make a device. The processing can be performed with one of several different techniques, such as with direct contact heating, such as with a salt bath or the use of electric current; heat applied from a distance, such as with a laser; with mechanical or thermal cycling; neutron irradiation; ultrasonic energy; or with some other ion treatment. The process can be performed in a computer controlled, automated manner, and can be used on wires or other shaped portions in the device, such as a planar shape.

Alternatively, different segments of the device can be bonded together, in which case it would typically be desirable, but not necessarily required, to provide a sleeve at joints where sections of the device are bonded together.

The present invention thus includes devices, including stents, septal occluders, blood clot filters, and guide wires, or parts of devices, such as wires used to make such devices, with portions having different recovery force characteristics from other portions, methods for selectively altering recovery force in desired locations of devices or parts of devices, and uses of such devices. Selected portions of the device, such as portions that are in contact with tissue or at the tissue/device interface, can be made to have different recovery forces. This ability can be used to reduce the force on certain tissues or in a vessel. Other features and advantages will become apparent from the following detailed description and drawings.

### BRIEF DESCRIPTION OF THE DRAWINGS

FIG. 1 is a perspective view of an exemplary a stent with portions with recovery force characteristics different from adjacent portions.

FIG. 2 is a plan view of a daisy occluder that can be treated according to the present invention.

FIG. 3 is a side view of a filter that can be treated according to another embodiment of the present invention.

FIG. 4 is a side view of an occluder that can be treated according to another embodiment of the present invention.

FIG. 5 is a patent foramen ovale (PFO) occluder that can be treated according to another embodiment of the present invention.

FIG. 6 is a guide wire that can be treated according to another embodiment of the present invention.

FIG. 7 is a block diagram of an automated system for modifying the recovery force characteristics portions of a device.

FIG. 8 is a side view of a wire in a holder for applying heat at desired locations.

FIG. 9 has graphs of materials processed as set out herein.

### DETAILED DESCRIPTION

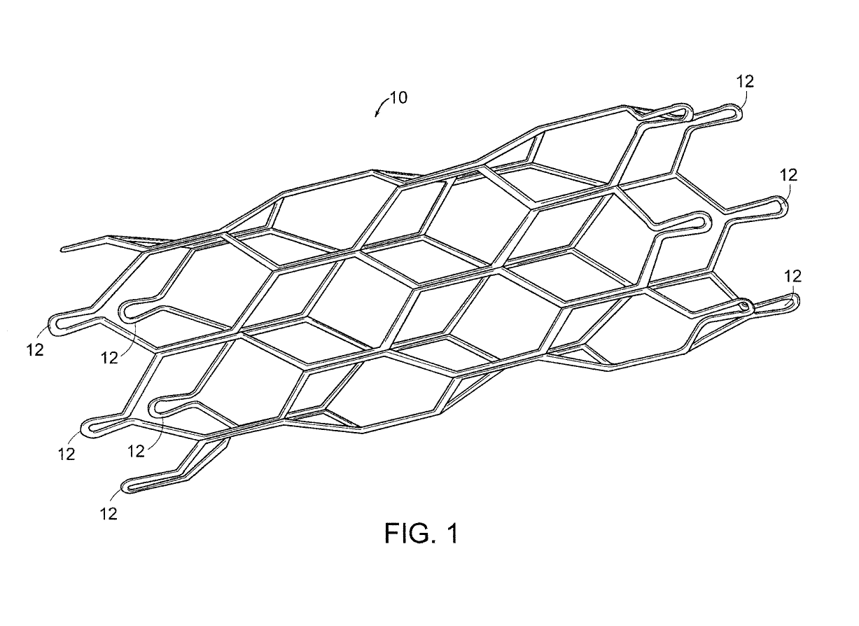

FIGS. 1-6 illustrate devices in which portions can be altered to have a lower recovery force, also referred to here as “softer” portions. The specific configurations of these devices are exemplary—there could be variations in the designs.

Referring to FIG. 1, for example, a stent 10 is a metal scaffold used to help hold open a portion of a vessel. As indicated in FIG. 1, which is taken from U.S. Pat. No. 5,540,712, there are looping fingers 12 at ends that would come into contact with the vessel. Because of the looping geometry, fingers 12 can be more rigid against the vessel. To reduce contact force to the vessel, fingers 12 or other desired portions can be made with less recovery force (and thus softer) by treating these sections. This treatment can be provided at both ends of the device, and can be done independently of the configuration of the vessel and regardless of any cross-section of the vessel.

The treatment can alter the crystal structure of fingers 12 of the stent to increase the transition temperature to the austenitic phase at the treated portions, while other portions have a lower transition temperature. The treatment can be applied to wires or other such parts in advance before such parts included in the device are formed into the desired stent shape, or the parts can be formed to make the stent and treated thereafter.

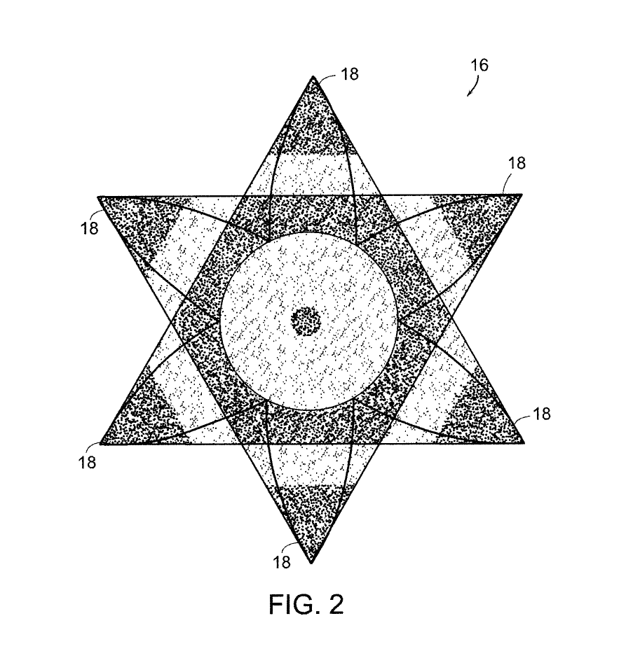

Other medical devices that can be treated in this manner are shown, for example, in FIGS. 2-6. FIG. 2 shows a daisy occluder 16 formed from a single length of wire with a tissue scaffold as shown in U.S. Pat. No. 5,741,297. Occluder 16 has loops 18 that come into contact with tissue when the device is used as a septal occluder; ends of these loops can be softened as desired, while other parts of the occluder are not softened.

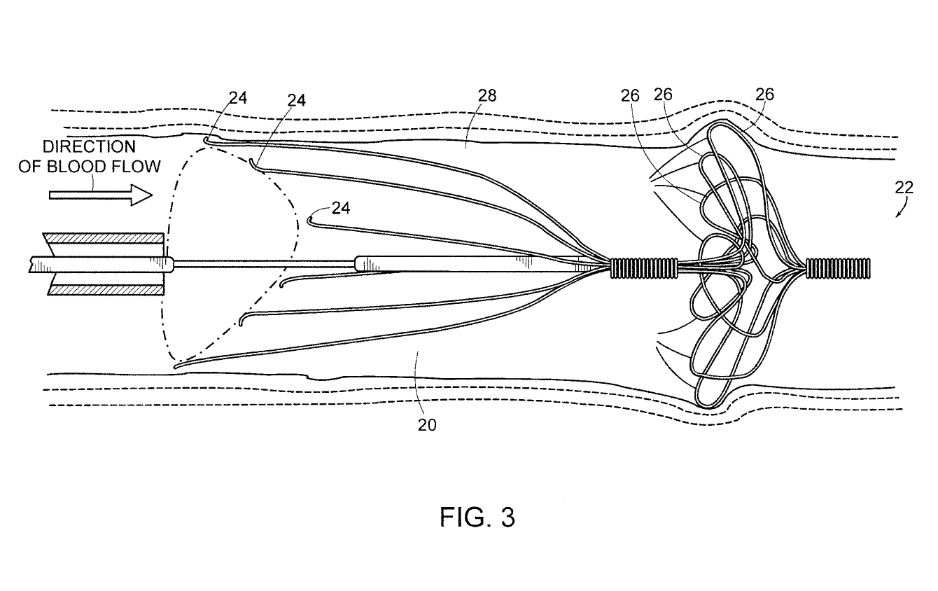

FIG. 3 shows a blood clot filter 22 inserted into a vein, as shown in U.S. Pat. No. 4,425,908. As shown here, filter 22 has seven lengths of wire, each with hooks 24 and loops 26 that contact the vein. In this example, midpoints (intermediate portions) of the wires leading to hooks 24, as shown by arrows 28, can be treated to be softened, thereby lessening the force with which hooks 24 contact the vessel. These portions 28 are where force is applied to the ends to contact tissue in the body.

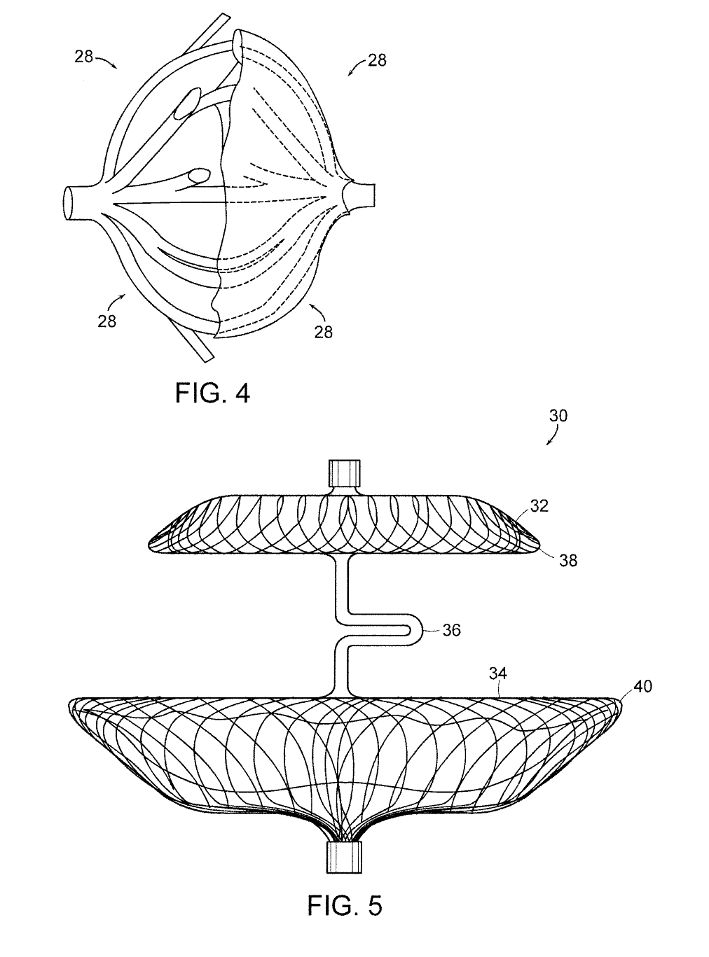

FIG. 4 shows an occluder from WO 0027292, with portions that could be softened indicated by arrows 28. As shown here and indicated in WO 0027292, spokes can be cut from a tube, and thus the portions with softened sections can have rectangular cross-section.

FIG. 5 is a patent foramen ovale (PFO) occluder 30 made from a continuous tubular metal fabric as described in U.S. Pat. No. 5,944,738. Two aligned disks 32, 34 are linked together with central portion 36. Portions 38 and 40 can be treated to soften them.

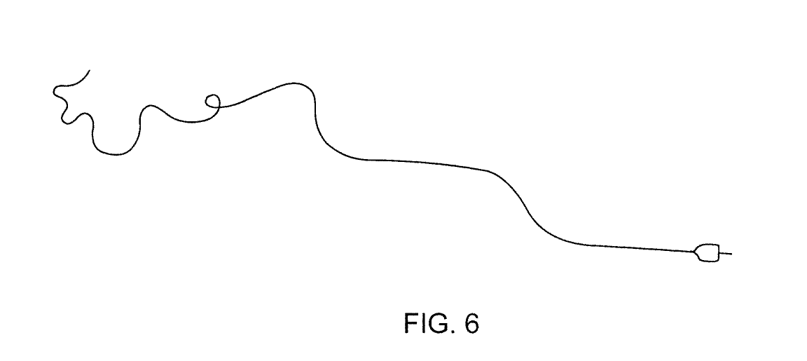

FIG. 6 shows a guide wire from U.S. Pat. No. 6,348,041. Selected sections of the wire can be treated to selectively alter recovery forces at desired portions, such as portions where the guide wire as inserted is more likely to contact a vessel. The softened portions could be at the end or in an intermediate area. For example, if it is know that the wire will extend to a particular location, and that within a given range of centimeters before the end there is a location where the guide wire will bend and contact a vessel, that intermediate portion may be softened such that portions on either side of the softened portion are stiffer.

While certain devices have been mentioned here, the treatments can be used for other devices or portions thereof, including, for example, for manipulator devices as described in U.S. Pat. No. 5,720,754.

An example of the processing of a portion of a device is described for a stent. In the case of the stent shown in FIG. 1, the device could be fabricated to the form shown in FIG. 1, and then could be placed on end into a hot liquid, such a salt bath at 430° C., for a desired period of time to soften the tips, while other portions of the device can be in contact with a heat sink to limit the heating to the desired areas. This process could then be repeated for the other end of the stent. The heating process alters the crystal structure of the desired portions of the device to increase the transition temperature to the austenitic phase in the treated portion relative to other portions.

Alternatively, one or more components used to make a device can be treated before being formed into the device, such as treating wires before they are formed into a device. Referring to the example of a laser, a laser could be mounted on a machine that moves along at least two coordinate axes (x and y), or that can move up to the six degrees of freedom (x, y, z, pitch, yaw, and roll). Tables for holding such devices to operate on work pieces are known in other fields, such as glue dispensing devices for circuit board processing and microarray printing onto slides for probe-target interaction. In each case, a controller can control movement of a device and its operational time to cause heat to be generated as desired locations for desired times.

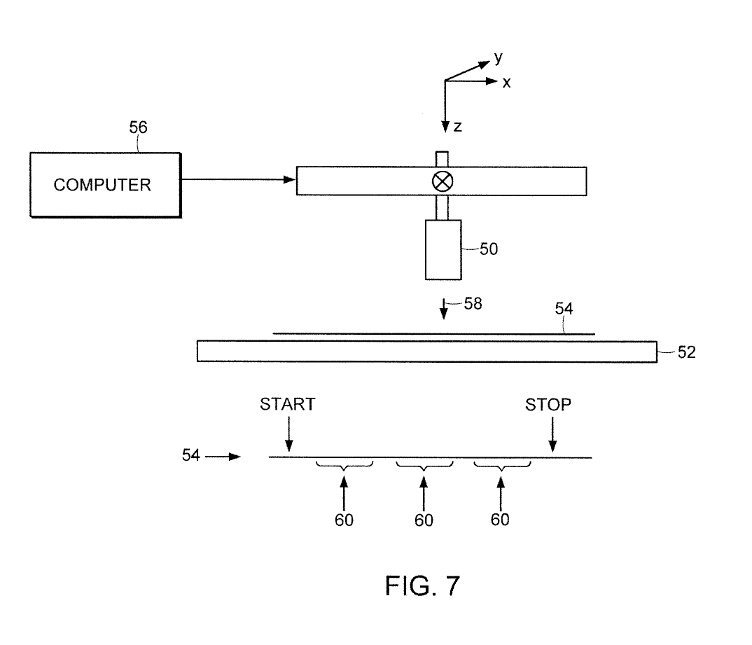

Referring to FIG. 7, a laser 50 is mounted over a table 52 and is movable along three coordinate axes (x, y, and z). One or more wires 54 are placed on table 52 at a known location. Using control from a computer 56, laser 50 can move from one region of wire 54 to the next to direct energy 58 to selected portions of wire 54. If the wire changes color under certain processing conditions, such as heat (as nitinol does), laser 50 can be used to create a marker at the beginning of the wire where it is being treated. For example, assume that wire to be used in a device needed to be 10 cm long with 1 cm soft sections at 2.5, 5.0, and 7.5 cm respectively from one end. The laser can be moved to a start point, direct heat sufficient to discolor the wire to create a reference start point of zero, then moved to create softer sections 60 at 2-3 cm, 4.5-5.5 cm, and 7-8 cm referenced from the start point. A stop point can also be defined. After these sections are created, the wire can be cut at the start and stop points, and the wire can then be processed as desired to produce the loops in desired locations. In the devices of FIGS. 2 and 3, for example, wires can be treated and then formed with the desired shape with softened sections at desired locations. Multiple wires could be treated on the work table at the same time.

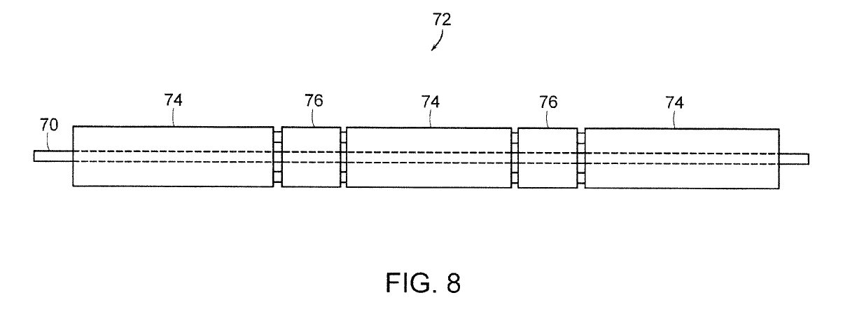

Referring to FIG. 8, in another embodiment for treating a wire 70, a holder 72 is provided for holding the wire and applying heat at selected locations. Holder 72 can have a hollow opening through its center or be hinged or in some other manner be opened to allow a wire to be positioned inside and then closed to hold wire 70. Holder 72 can have at least two types of sections 74 and 76. Sections 76 can be coupled to a heat source, while sections 74 are coupled to heat sinks. The heat source provides heat through coils or some other heating method that allows the heat to be localized to desired portions of the wire.

With the system of the type as shown in FIG. 8, sections for applying or sinking heat can be created and moved so that the wire can be treated before being bent into a desired shape. With many such holders, or with a holder that has multiple channels for wires, wires can be processed on a larger scale.

The result is a wire or other shaped part that can have a uniform diameter, and is made from one material, but with sections that have different properties and that may be short in length and between other sections with more rigid properties. The device may have an appearance that does not indicate where the softer sections are, or the sections may be identified or identifiable, such as if there is a color difference. A wire can have a regular cross-section, such as circular or square, or an elongated cross-section such as a rectangular cross-section, as in FIG. 4, or any other regular or other polygon. It can be much thinner than it is wide, and thus appear as a sheet or even a film. A wire can be solid, hollow and tubular, or have more than one co-axial layer of material.

Similar to a laser, devices for providing ultrasonic energy or ion bombardment can be mounted and selectively directed to desired portions of a device or component used to make a device. The wire could be wrapped in a coil in selected locations, although such a process would be difficult to automate without additional structures.

While the treatments have been described above as being made to a relatively stiffer wire to make it softer, treatments could be applied to a softer wire to make desired sections stiffer, so that in either case the net result is a continuous wire that has different flexible properties in the alloy itself.

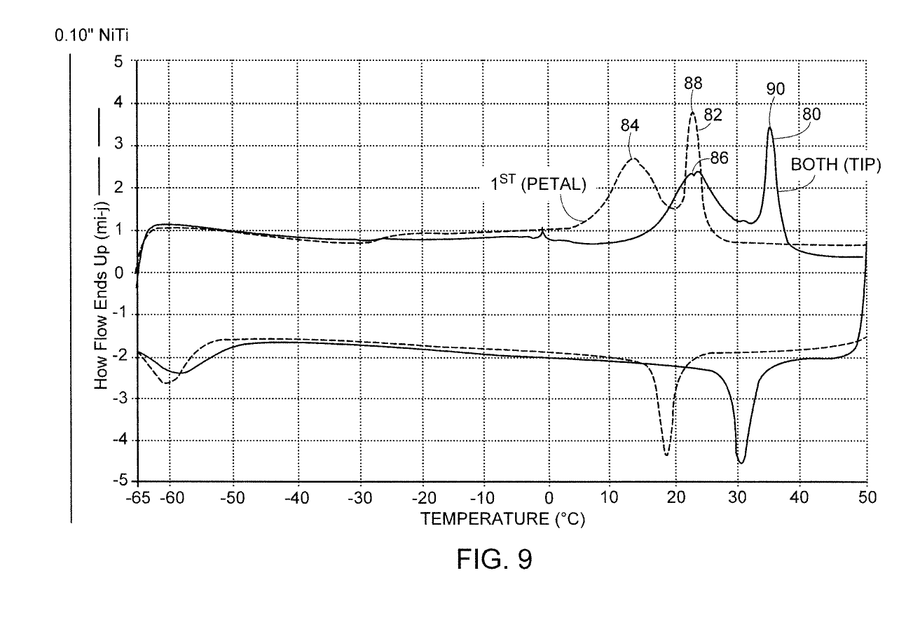

FIG. 9 has graphs demonstrating the heat flow of two identical wires that were processed differently to produce different transition temperatures. These plots are made using a differential scanning calorimeter (DSC), a known device used to measure transition temperatures in materials.

The plots have two curves 80 and 82, with the top part of the curves showing heat flow as a function of temperature as the wire is heated, and the lower curve as the wires are cooled. A wire was annealed at 500° C. for 25 minutes; a piece was removed and the heat flow measured, resulting in curve 82. The remaining wire was further annealed at 430° C. for 60 minutes, and the heat flow was measured, resulting in curve 80. The curves reach a first R′-phase peak (R′p) at 84 and 86, which shows where the crystal structure of the wires changes from a martensite phase to an R-phase. The peak is where the material is in transition and is about 12° C. wide. At peaks 88 and 90, the material transitions to the austenitic phase. The peak is sharper for this transition with a width of less than about 5° C.

Thus the wire that was annealed twice has transition peak temperatures, R′p and Ap, that are about 12° C. higher than the corresponding peaks for the wire that was annealed once. If the device is to be inserted in a body, the body will have a body temperature. Wires or portions of wires can be treated so that all portions are in the austenitic phase at body temperature. The part with the lower transition temperatures will have greater recovery force.

Alternatively, the device can be treated so that portions are in the austenitic phase and other portions are in the R-phase at body temperature, in which case the portions in the austenitic phase at body temperature will typically have greater recovery force.

It is desirable for the difference in the corresponding austenitic peaks Aₚ (see ASTM F2005-00, FIG. 1 and FIG. 2) to be at least about 5° C. apart, and preferably at least the width of the peak (as measured, e.g., by a DSC). It is also desirable for the difference in the corresponding R′-phase peaks to be at least about 5° C. apart. In the example of FIG. 8, the austenitic peaks are about 5° C. wide, so 5° C. would be a sufficient difference. If the peaks were about 10° C. wide, it might be desirable to have a greater difference closer to 10° C. Note that the ASTM standard referred to above uses the term “transformation temperature,” but that term has the same meaning as “transition temperature.”

Having described embodiments of the invention, it should be apparent that modifications can be made without departing from the scope of the appended claims. For example, aspects of the present invention can be used with many types of medical devices, including stents, septal occluders, left atrial appendage (LAA) closure devices, or blood clot filters with softened sections at certain desired locations, as well as guide wires, needles, catheters, cannulas, pusher wires, and other components of delivery or recovery systems for implants, such as stents or filters. These different portions with different recovery force are preferably made of the same material and same cross-section. There can further be multiple sections, each with different recovery force if desired. While recovery force is discussed, the invention can be used to increase stiffness without fully recovering to a desired shape.

What is claimed is:

1. A medical device made of at least one wire having first, second, and third sequential sections, the first, second, and third sequential sections having the same material, the first and third sections having a first transition peak temperature, and the second section having a corresponding second transition peak temperature that is greater than the first transition peak temperature by at least about 5° C.

2. The device of claim 1, wherein the first, second, and third sequential sections are made of nitinol.

3. The device of claim 2, wherein the first and second transition peak temperatures are austenitic peak temperatures.

4. The device of claim 2, wherein the first and second transition peak temperatures are R′-phase peak temperatures.

5. The device of claim 1, wherein the second transition peak temperature is greater than the first transition peak temperature by at least about 10° C.

6. The device of claim 5, wherein the second transition peak temperature is greater than the first transition peak temperature by at least about 12° C.

7. The device of claim 1, wherein the wire is solid.

8. The device of claim 1, wherein the wire is tubular.

9. The device of claim 1, wherein the wire has a polygonal cross-section.

10. The device of claim 1, wherein the wire has a rectangular cross-section.

11. The device of claim 1, wherein the first, second, and third sections form a loop designed to contact tissue of a patient when the device is deployed, the curved portion of the loop being in the second section.

12. The device of claim 11, wherein the device is a stent having multiple loops at an end of the stent, each loop having a curved section with a recovery force that is less than the recovery force of adjacent sections.

13. The device of claim 11, wherein the device is a blood filter.

14. The device of claim 11, wherein the device is an occluder.

15. The device of claim 14, wherein the occluder has a plurality of loops, each of which has a curved section with a recovery force that is less than the recovery force of adjacent sections.

16. The device of claim 1, wherein the device is a guide wire.

17. The device of claim 1, wherein the device includes a spoke having an end for contacting tissue of a patient, the end being part of the third section.

18. A method for making a medical device including treating a wire that is part of the medical device and that has first, second, and third sequential sections with the first, second, and third sequential sections being made of the same material, the treating being provided so that the first and third sections having a first transition peak temperature, and the second section has a corresponding second transition peak temperature that is greater than the first transition peak temperature.

19. The method of claim 18, wherein the first, second, and third sequential sections are made of nitinol.

20. The method of claim 19, wherein the transition peak temperatures are austenitic peak temperatures.

21. The method of claim 19, wherein the transition peak temperatures are R′-phase peak temperatures.

22. The method of claim 18, wherein the first, second, and third sections form a loop, with the curved portion of the loop being in the second section.

23. The method of claim 22, wherein the device is a stent having multiple loops at an end of the stent, each loop having a curved section with a recovery force that is less than the recovery force of adjacent sections.

24. The method of claim 22, wherein the device is a blood filter.

25. The method of claim 22, wherein the device is an occluder.

26. The method of claim 18, wherein the device is a guide wire.

27. The method of claim 18, wherein the device includes a spoke having an end for contacting tissue of a patient, the end being part of the third section.

28. The method of claim 18, wherein the treating includes providing a heat treatment to the second section different from a treatment provided to the first and second sections.

29. The method of claim 28, wherein the heat is applied by direct contact to the wire.

30. The method of claim 29, wherein the heat is applied with a hot liquid bath.

31. The method of claim 28, wherein the heat is applied by an energy source not in direct contact with the wire.

32. The method of claim 31, wherein the energy source is a laser.

33. The method of claim 28, wherein the heat is applied by an energy source with a computer controlled positioning system.

34. The method of claim 28, wherein the heat is applied to the wire while the wire is in a first shape, the method further comprising bending the wire into a shape suitable for use in the medical device after the treating.

35. The method of claim 34, wherein the heat is applied to the second section while the first, second, and third sequential sections are in a straight line, and thereafter bending the wire to form a loop with the curved portion in the second section.

36. The method of claim 28, wherein the heat is applied in an automated manner.

37. The method of claim 28, wherein the heat is applied with a coil.

38. The method of claim 18, wherein the treating includes using one of ion bombardment and ultrasonic energy.

39. The method of claim 18, wherein the device is treated so that the second transition peak temperature is greater than the first transition peak temperature by at least about 5° C.

40. The device of claim 39, wherein the second transition peak temperature is greater than the first transition peak temperature by at least about 10° C.

41. The device of claim 40, wherein the second transition peak temperature is greater than the first transition peak temperature by at least about 12° C.

42. A medical device for insertion into a patient, the device having one or more wires with a plurality of loops circumferentially arranged and having curved portions for contacting tissue of a patient, wherein the curved portions for contacting the tissue of the patient have a transition peak temperature that is greater than portions immediately adjacent to the curved portions by at least 5° C.

43. The device of claim 42, wherein the device is a septal defect occluder formed from a single wire.

44. The device of claim 42, wherein the device is an occluder formed from a plurality of wires with loops.

45. The device of claim 42, wherein the device is a filter formed from a plurality of wires with loops.

46. The device of claim 42, wherein the device is a stent a plurality of loops at an end of the stent.

47. The device of claim 42, wherein the device is made of nitinol.

48. The device of claim 42, wherein the transition peak temperature is the austenitic peak temperature.

49. The device of claim 42, wherein the transition peak temperature is the R′-phase peak temperature.
